(19) United States
(12) Patent Application Publication
(10) Pub. No.: US 2026/0053447 A1
(43) Pub. Date: Feb. 26, 2026

(54) METHOD AND SERVER COMPUTER FOR FINDING A TIMING OF COMMUNICATION WITH RESPECT TO PHYSIOLOGICAL INFORMATION AND ASSOCIATED TIMESTAMPS

(71) Applicants: Kura Care, INC., San Diego, CA (US); KURA, Med Inc., Taipei City (TW)

(72) Inventors: Ming-Tse Tsai, Poway, CA (US); Chiih-Wei Chiu, Kaohsiung City (TW); Hao-Chun Chang, New Taipei City (TW); Jake Bramwell, Hauula, HI (US); Kai-Chieh Yang, Taoyuan City (TW)

(21) Appl. No.: 19/306,441

(22) Filed: Aug. 21, 2025

Related U.S. Application Data
(60) Provisional application No. 63/687,005, filed on Aug. 26, 2024.

(51) Int. Cl. A61B 5/00

(52) U.S. Cl. CPC A61B 5/7282; A61B 5/0022; A61B 5/7264; A61B 5/7275

(57) ABSTRACT

A method for finding a timing of communication with respect to physiological information and associated timestamps is provided. The method comprising: receiving physiological measurement information of a first user from a sensing device via a network; obtaining a first timing of communication of first health specific information according to the physiological measurement information and corresponding timestamp; and transmitting the first health specific information to a client computer at the first timing.

### CROSS REFERENCE TO RELATED PATENT APPLICATION

This patent application is based on a provisional patent application No. 63/687,005 filed on Aug. 26, 2024.

### FIELD OF THE INVENTION

The present invention relates to a network system, and more particularly, to a network system for notifying patients health specific information at calculated timings according to their physiological measurements and corresponding timestamps.

### BACKGROUND OF THE INVENTION

Patients suffer from memorizing all lifestyle instructions from their doctors or any other authoritative sources and fail often to implement these instructions as tasks in their daily life. The failures may be resulted from few reasons. First, these instructions may be difficult to follow because the timings for implementing instructions may conflict with other daily activities of patients. Second, the instructions may be responsive to some kinds of physiological measurements. It requires a notification mechanism to deliver the reactive instructions to patients in time.

A patient was instructed to take antihypertensive pill after breakfast at the same time daily to control his blood pressure whole day. However, the patient is required to get up earlier some days, e.g., Monday and Friday, in a week to go to work. While he wakes up in the dawn and rushes to his work, he ends up missing his breakfast and the pill frequently, which results in bad blood pressure control. This reflects that other daily activities of patient may conflict with the instruction following.

Say for the same patient described above, in one day, he forgot to take the antihypertensive pill and breakfast. He works until 11:00 am then he takes a lunch break. During the break, he takes a blood pressure measurement, and it reads 160/100 mmHg. He thinks to himself, “should I take my antihypertensive pill now?” Without timely instruction, the patient is left puzzled and very likely to continue afternoon work without taking any action on his blood pressure. That means the instruction should be timely provided and responsive to the physiological measurement.

Say for the same patient described above, he had a stomach bug for days and nearly had nothing. This morning, he got up at the usual time. Although he did not have an appetite for breakfast, he did remember a pill waits for him. Nevertheless, he took a blood pressure measurement, and it shows 100/50 mmHg, which was much lower than his usual blood pressure. He then thought to himself, “should I take my antihypertensive pill now?” Again, this also teaches the instruction should be timely provided and responsive to the physiological measurement.

From the three exemplary scenarios, daily activities might come into play in a patient's daily schedule and interfere with the patient's decision. And when making decisions after taking physiological measurement, the patient wishes to have timely instructions.

Thus, there exists a need to provide an instruction informing mechanism to patients at appropriate timings to meet their feasible options and to respect their decisions.

### SUMMARY OF THE INVENTION

According to an embodiment of the present application, a method for finding a timing of communication with respect to physiological information and associated timestamps is provided. The method comprising: receiving physiological measurement information of a first user from a sensing device via a network; obtaining a first timing of communication of first health specific information according to the physiological measurement information and corresponding timestamp; and transmitting the first health specific information to a client computer at the first timing.

In one embodiment of the present application, a server computer for finding a timing of communication with respect to physiological information and associated timestamps is provided. The server computer comprising: a networking device connected to a sensing device and a client computer via a network; a processor configured for executing instructions stored in non-volatile memory for realizing following: having the networking device receive physiological measurement information of a first user from the sensing device; obtaining a first timing of communication of first health specific information according to the physiological measurement information and corresponding timestamp; and having the networking device transmit the health information specific information to a client computer at the first timing.

According to an embodiment of the present application, a network system for finding a timing of communication with respect to physiological information and associated timestamps is provided. The network system comprises the server, the sensing device, and the client computer.

Based on the method and the server provided in the present application, an instruction informing mechanism for notifying patients or users at appropriate timings to meet their feasible options and to respect their decisions are presented. The mechanism can automatically analyze the periodic schedule and timings of communication based on the physiological measurement information and corresponding timestamps. Hence, health specific information such as reminder, an action suggestion, and/or a summary of physiological measurement information can be informed at the timings of communication in which the mechanism finds. It helps the patients or the users to follow the instructions in the timings of communication which do not conflict with other activities of the user. And the responsive report of physiological measurement information can be delivered to the patients or the users in appropriate timings. Following this mechanism, the user may make a better choice proactively and reflectively because the mechanism provide right information to the user at the right time.

### BRIEF DESCRIPTION OF THE DRAWINGS

The advantages and spirit related to the present invention can be further understood via the following detailed description and drawings.

FIG. 1A illustrates a block diagram of a network system 100 for informing user at calculated timings in accordance with an embodiment of the present application.

FIG. 1B illustrates a variant of the network system 100 as shown in FIG. 1A for informing user at calculated timings in accordance with another embodiment of the present application.

FIG. 1C illustrates a variant of the network system 100 as shown in FIG. 1A for informing user at calculated timings in accordance with another embodiment of the present application.

FIG. 2 depicts a block diagram of a computer 200 in accordance with an embodiment of the present application.

FIG. 3 illustrates a flowchart diagram of a method 300 for finding timings to transmit health specific information in accordance with an embodiment of the present application.

FIG. 4 illustrates a flowchart diagram of a method 400 for finding a pattern between the timing of communication and its corresponding measurements in accordance with an embodiment of the present application.

FIG. 5 illustrates a flowchart diagram of a method 500 for finding timings to transmit health specific information in accordance with an embodiment of the present application.

FIG. 6A illustrates a flowchart diagram of a method 600 for finding timings to transmit health specific information in accordance with an embodiment of the present application.

FIG. 6B illustrates a variant of the flowchart diagram of the method 600 as shown in FIG. 6A.

FIG. 7 illustrates a flowchart diagram of a method 700 for finding timings to transmit health specific information in accordance with an embodiment of the present application.

FIG. 8 depicts a method 800 for finding timings to transmit health specific information in accordance with an embodiment of the present application.

### DETAILED DESCRIPTION OF THE PREFERRED EMBODIMENT

Some embodiments of the present application are described in details below. However, in addition to the description given below, the present invention can be applicable to other embodiments, and the scope of the present invention is not limited by such rather by the scope of the claims. Moreover, for better understanding and clarity of the description, some components in the drawings may not necessary be drawn to scale, in which some may be exaggerated related to others, and irrelevant. If no relation of two steps is described, their execution order is not bound by the sequence as shown in the flowchart diagram.

One of the perspectives of the present application is to provide a network system for informing user at calculated timings. The network system collects physiological measurement information (may be referred as physiological measurement, measurement information, or physiological information in the present application) from the user by a sensing device; generating health specific information for the user based on the physiological measurement information and corresponding timestamps to remind available actions or to review what happened and finding a timing of communication for delivering the health specific information to the user's computer (smartphone, smart band; and/or smart watch); and collecting the user's response about the timing; and based on the responses, reiterates the process to refine the timing finding strategy.

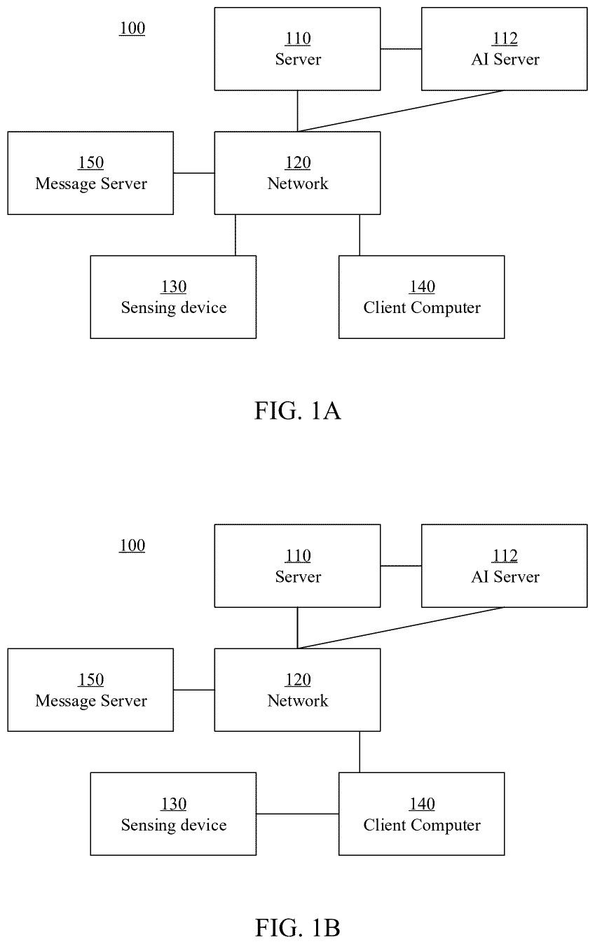

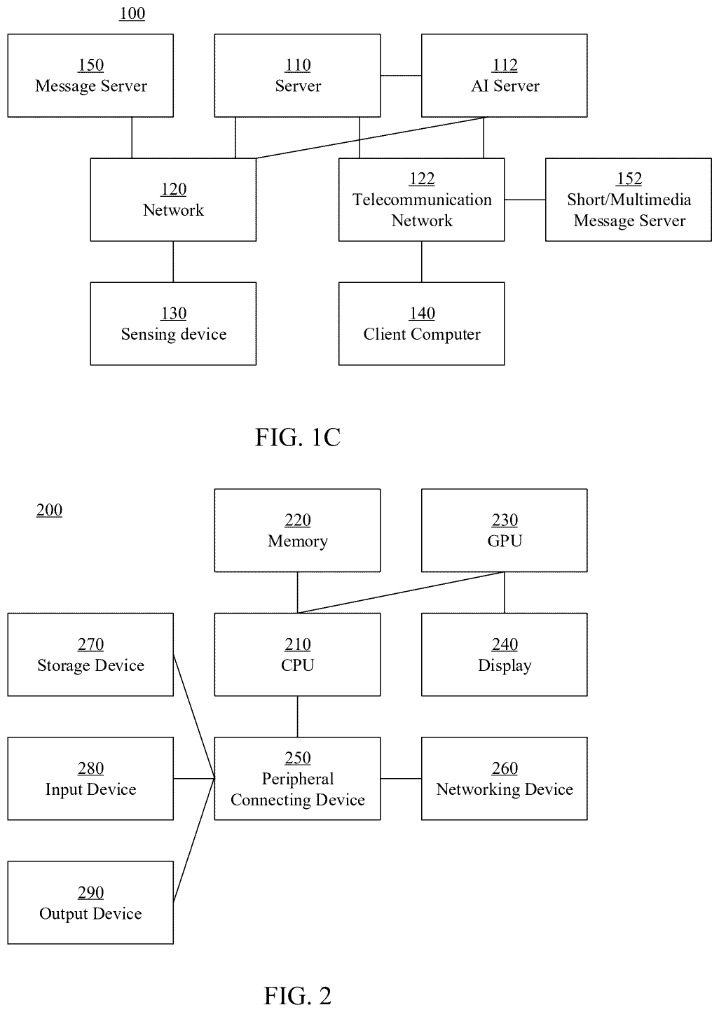

Please refer to FIG. 1A, which illustrates a block diagram of a network system 100 for informing user at calculated timings in accordance with an embodiment of the present application. The network system 100 may comprise a server 110, a network 120, a sensing device 130 for a user, a client computer 140 for the user, an optional AI (Artificial Intelligence) server 112, and an optional message server 150.

The network 120 is configured for carrying data exchanges between the server 110 and the sensing device 130 and data exchanges between the server 110 and the client computer 140. For example, the network 120 may comprise access networks such as WiFi/IEEE 802.11 networks, 3G/4G/5G/6G compliant networks, IEEE 802.3 networks, PSTN networks, optical fiber networks, and/or xDSL networks. The server 110, the sensing device 130, the client computer 140, the optional AI server 112, and/or the message server 150 may be configured to connect to the access networks. Moreover, the network 120 may also comprise backbone networks, e.g., telecommunication networks, for connecting the access networks.

The sensing device 130 is configured to measure the user's physiological signatures such as glucose, blood pressures, heart rate, respiration rate, blood oxygen rate, and/or electrocardiographic measurements. Thus, the sensing device 130 includes one or more specific sensors to measure the user's physiological signatures and a networking device for connecting the network 120.

In one example, the sensing device 130 adds a local timestamp to the measurement information before transmitting it to the server 110. The clock kept by the sensing device 130 may be synchronized with the server 110 or a common source such as a timing service provider. Therefore, the timestamp included in the measurement information received by the server 110 does synchronize with the server's internal clock.

In another example, the sensing device 130 does not add its local timestamp to the measurement information. Instead, the server 110 would generate a timestamp according to its internal clock for the received measurement information. Of course, the server 110 can always generate a timestamp according to its internal clock for the received measurement information which may be with or without an attached timestamp.

The transmission of the measurement information may be pass through the message server 150. The message server 150 may implement one or more public message service for the server 110 and the sensing device 130. For example, the message server 150 may be an electronic mail server, an instant message server (like Line, WhatsApp, Skype, Facetime etc.,) or a message queue server. One of the functions provided by the message server 150 is asynchronous transmission. It means that the message, i.e., the measurement information, can be temporarily stored in the message server 150 and be resent by the message server 150 until it is safely received by the server 110. Another one of the functions provided by the message server 150 is encrypted transmission to guarantee the safety of the transmission. However, the message server 150 is optional in the embodiment of the present application. The sensing device 130 may transmit the measurement information of the user to the server 110 without the intervention of the message server 150.

After receiving several physiological measurement information from the sensing device 130, the server 110 may analyze the physiological measurement information and their corresponding timestamps to obtain the user's behavior or periodic schedule of the user. The accumulated timestamps and the corresponding physiological measurements can be used to infer the user's daily tasks. The daily or weekly tasks may include taking meals (breakfast, lunch, afternoon tea, dinner), exercises, works, and sleep. A collection of tasks in one day constitutes a behavior or a daily schedule of the user. Similarly, a collection of tasks in one week or in one month constitutes a behavior or periodic schedule of the user. In one embodiment, the analysis of the physiological information and corresponding timestamp may be done by inferencing one or more machine learning algorithms/models to obtain a periodic schedule of the user. The periodic schedule is consisted of at least one task. One or more kinds of physiological measurement information may be taken into considerations in the analysis.

In one embodiment, a first machine learning algorithm/model is used to infer a timing of communication based on the physiological measurement information and corresponding timestamp of the user. In an alternative embodiment, a second machine learning algorithm/model is used to infer the timing of communication based on a periodic schedule of the user. And the periodic schedule of the user is the analysis result of the physiological measurement information and corresponding timestamp of the user. The analysis may also take advantage of a third machine learning algorithm/model to infer the periodic schedule of the user based on the physiological measurement information and corresponding timestamp of the user.

When these tasks (included in the behavior and the periodic schedule) and the associated timings are available, task-specific health specific information may be sent to a client computer of the user either to remind him of what to do with an actionable plan or to review with him what happened. Following this way, the user may make a better choice proactively and reflectively because the network system 100 provide right information to the user at the right time.

The server 110 may perform the analysis corresponding to the measurement information and timestamps of the user by itself. In an alternative embodiment, the AI server 112 may be further utilized by the server 110 to generate the task-specific health specific information of the user. In one example, the AI server 112 may be configured to train or to infer various machine learning algorithms/models which are used in the embodiments of the present application. The machine learning models may be a combination of various neural networks such as convolutional neural networks, deep neural networks, and transformer networks etc. Person having ordinary skill in the art can understand the AI server 112 may be provided by vendors as a cloud service. For examples, Google's cloud service, Amazon's AWS service, and Microsoft's Azure service etc. provide various kinds of neural network services which can be considered as the AI server 112 in the present application. However, without the AI server 112, the server 110 may have enough computing power to implement the machine learning algorithms/models presented in the application.

The task-specific health specific information may include the actionable plan and/or the review of measurement information. For examples, the actionable plan may include advice of exercises, rests, sleeps, and/or diet suggestions. The review of the measurement information may include charts of physiological information and explanations. Moreover, the review may include a survey of the task. For example, a sleep quality survey after a sleep.

In one embodiment, parts of the content of task-specific health specific information may be generated by LLMs (large language models) implemented by the AI server 112. Based on the measurement information and the corresponding timestamps, the server 110 may compose or provide appropriate prompts to LLM implemented by the AI server 112 and receive the health specific information from the AI server 112. For examples, the LLM may be something like commercially available products such as ChatGPT, DeepSeek, Perplexity, Co-pilot and/or Gemini.

Similarly, one or more machine learning models may be utilized to learn and to infer when to send the health specific information to a user. The machine learning model may be one combination of multiple neural networks. Initially, a general machine learning model may be used to output at least one timing to send health specific information to the specified user. After collecting the user's responses, a tailor-made machine learning model specifically for the user may be derived, fine-tuned, and/or retrained from the general machine learning model based on the user's responses to the health specific information. The general machine learning model may be also derived, fine-tuned, improved, refined and/or retrained based on a plurality of users'responses to the health specific information.

Once the health specific information and the timing are available, the server 110 is configured to send the health specific information at the timing to the user. The transmission of the health specific information may be end-to-end from the server 110 to the client computer 140 of the user via the network 120. Because the transmission is synchronized, the server 110 is able to know whether the health specific information is delivered to the client computer 140 in time. The client computer 140 may be smartphone, wearable electronics like smartwatch or smart goggle, tablet computer, laptop computer, and/or any computer which connects to the network 120.

Alternatively, the transmission of the health specific information may be completed with the intervention of the message server 150. Since the transmission is asynchronous, the server 110 may be also notified by the message server 150 when the client computer 140 receives the health specific information.

The user may respond to the health specific information with his opinions of the timing. For examples, the user may express one of two options, good and no-good, in view of the timing. In another example, the user may give a score from 1 to 10 of the timing. The user's response can also send back to the server 110 from the client computer 140. Given the timing matches with the receiving time of the client computer 140, the server 110 can pair the timing of the health specific information with the associated user's response. In other words, the timing of the health specific information is labeled by the user's response as a sample of the machine learning model.

Person having ordinary skill in the art of AI can understand the sample can be further used to derive, fine-tune, improve, refine and/or retrain the general machine learning model and/or the tailor-made machine learning model for the user. In next iteration, the tailor-made machine learning model can be used to infer the timing of next health specific information based on the measurement information(s) and the associated timestamp(s) of the specified user.

Please refer to FIG. 1B, which illustrates a variant of the network system 100 as shown in FIG. 1A for informing user at calculated timings in accordance with another embodiment of the present application. Comparing with the network system 100 as shown in FIG. 1A, the sensing device 130 as shown in FIG. 1B does not direct connect to the network 120. Instead, the sensing device 130 connects to the server 110 via the client computer 140 of the user.

For example, the sensing device 130 may be a wearable device which connects with the client computer 140 via short ranged wireless communication such as Bluetooth, NFC (Near Field Communication) or wired communication such as USB, Firewire, and UART (RS-232/422). An application program or a driver program corresponding to the sensing device 130 may be configured to receive the measurement information from the sensing device 130 and to transmit the measurement information with or without timestamps to the server 110 via the network 120. Like abovementioned embodiments, the transmission of the measurement information may be intervened by the message server 150 or end-to-end from the client computer 140 to the server 110.

Please refer to FIG. 1C, which illustrates a variant of the network system 100 as shown in FIG. 1A for informing user at calculated timings in accordance with another embodiment of the present application. Comparing with the network system 100 as shown in FIG. 1A, the server 110 communicates with the sensing device 130 and the client computer 140 via different networks, respectively.

In one embodiment, this network system 100 as shown in FIG. 1C may be applied to remote monitoring. It means that the sensing device 130 is configured to measure physiological information of a first user and the client computer 140 is configured to report to a second user. For example, the first user may be a patient; and the second user is a caretaker of the patient.

To send the health specific information to the client computer 140, in case which is a smartphone or a feature phone, the server 110 may take advantage of SMS/MMS (Short Message Service/Multimedia Message Service) provided by telecom operators. Thus, the server 110 may send the health specific information to the SMS/MMS server 152 which forward the health specific information to the client computer 140. Reversely, the user of the client computer 140 may also take advantage of the SMS/MMS server 152 to respond to the server 110 with respect to the timing of the health specific information.

The SMS/MMS server 152 may provide a scheduling function to the server 110. It means that the server 110 can upload the health specific information and set the timing of transmitting the health specific information in advance. Thus, when the preset time comes, the SMS/MMS server 152 can deliver the health specific information which is packed in the short message or the multimedia message to the client computer 140 via the telecommunication network 122.

Please refer to FIG. 2, which depicts a block diagram of a computer 200 in accordance with an embodiment of the present application. The computer 200 may be embodied as the server 110, the AI server 112, the sensing device 130, the client computer 140, the message server 150, and/or the SMS/MMS message server 152. Some components of the computer 200 may be absent or altered to fit the role it plays. For example, the AI server 112 may be lack of display 240 and input device 280 such as keyboard and mouse. The GPU (Graphics Processing Unit) 230 of the AI server 112 may be altered to dozens of NPUs (Neural network Processing Units.)

The computer 200 comprises at least one CPU (central processing unit) 210, a memory module 220 for system operation, an optional GPU 230 for generating content to be shown on a display 240, a peripheral connecting device 250 implementing industrial standard interfaces such as PCI, PCI-Express, SCSI, SATA, USB etc., a networking device 260 for connecting to the network 120 and/or the telecommunication network 122, a storage device 270 for storing operating system, application programs, and data for implementing the steps provided by the present application, one or more input devices 280, and one or more output devices 290. A operating system run by the CPU 230 is configured to control the computer 200. And various driver programs and application programs under the operating system may be used to implement the steps of the embodied methods provided by the present application.

In one embodiment, the sensing device 130 may have specified sensor as one of its input devices 280 for measuring user's physiological features including but not limited to glucose, blood pressures, heart rate, respiration rate, blood oxygen rate, and/or electrocardiographic measurements. The client computer 140 may have display 240 or touch screen for showing the health specific information and/or have a speaker for reading the health specific information. Moreover, the client computer 140 may have input devices 280 such as touch screen, touch panel, keyboard, or buttons to receive the user's response.

The server 110, the AI server 112, the message server 150, and/or the SMS/MMS server 152 may be consisted of a cluster of multiple physical or virtual computers 200 to enhance its computing capabilities. Person having ordinary skill in the art may be familiar with the knowledge of computer architecture and computer organization to understand the variants of the computer 200 as shown in FIG. 2 may be applied as various computers as shown in FIGS. 1A through 1C.

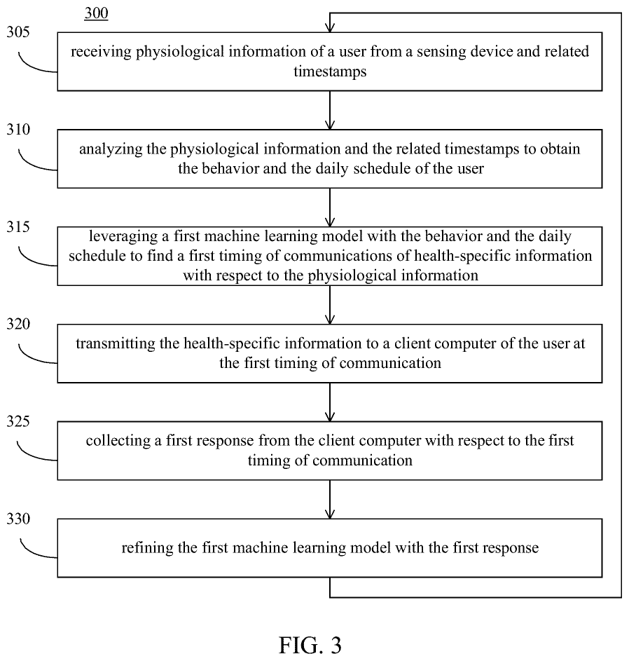

Please refer to FIG. 3, which illustrates a flowchart diagram of a method 300 for finding timings to transmit health specific information in accordance with an embodiment of the present application. The method 300 may be realized by the network system 100 as shown in FIG. 1. Especially, the method 300 may be embodied as computer instructions and data being executed by the server 110 as shown in FIG. 1. If no casual relation between two steps as shown in FIG. 3, the present application does not limit the execution sequence of the two steps. The method 300 may begin at step 305.

Step 305: receiving physiological information of a user from a sensing device and related timestamps. The related timestamps may be included in the physiological information sent from the sensing device or generated locally.

Step 310: analyzing the physiological information and the related timestamps to obtain the behavior and/or the daily schedule of the user and to generate health specific information of the user.

Step 315: leveraging a first machine learning model with the behavior and/or the daily schedule of the user to find a first timing of communication of the health specific information with respect to the physiological information.

The first machine learning model includes a set of methods to extract information from the needed information from the input information. Depending on the multiplicity of input variables and the predicted output, a method is chosen to learn a pattern from the input data, which is called “model”. And then this model will be applied for any new incoming input variables to get the predicted output.

In one example, the behavior and/or daily schedule includes a user's one or more upload times as input data to the first machine learning model. The first machine learning model may be trained to output the first timing (e.g. the most common time) based on a “k-mean” method applying to the upload times.

A Person having ordinary skill in the art can understand the first machine learning model may be multi-modal which takes different kinds of input data in other scenarios. In the example describe above, the upload times of the user can be used as the sole input data. However, the first machine learning model can take different kinds of input data other than upload times.

Step 320: transmitting the health specific information to a client computer at the first timing.

Step 325: receiving a first response from the client computer with respect to the first timing.

In one embodiment, the first response collected may include an uploading or receiving metadata (such as timestamp) corresponding to the first response. The timestamp as well as the content of the first response may be further inputted into the first machine learning model or an algorithm to calculate a mean time of the first timing for communicating with the user.

Step 330: refining the first machine learning model with the first response.

For example, a user interacts with the server 110 for several months, e.g., from June 2025 to December 2025. Hence, there are many sets of the physiological information, the daily schedule, the behavior, the health specific information, and first responses and their corresponding timings and/or timestamps in the records. A part or all of these records may be treated as input data variables to the first machine learning model. The first machine learning model may include a “k-mean” method so that a best time or the most common time can be informed by the first machine learning model.

On one day after the interactions of the specified user, e.g., the first day of 2026, the records collected during June to December, 2025 are applied to the first machine learning model to find out a latest version of first timing of communication with the user.

Although records collected during several months including many tuples of first responses can be used as input data in the described example above, the first machine learning model can be refined by only one tuple of first response in an alternative example. In other words, the refining step can be performed each time the method 300 is executed. Or the refining step can be performed once after several iterations of the method 300 are executed.

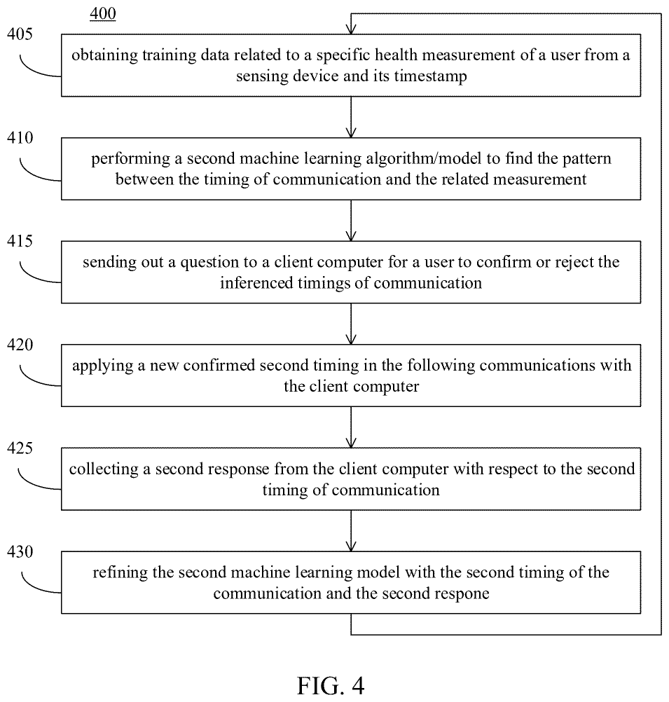

Please refer to FIG. 4, which illustrates a flowchart diagram of a method 400 for finding a pattern between the timing of communication and its corresponding measurements in accordance with an embodiment of the present application. The method 400 may be realized by the network system 100 as shown in FIG. 1. Especially, the method 400 may be embodied as computer instructions and data being executed by the server 110 as shown in FIG. 1. If no casual relation between two steps as shown in FIG. 4, the present application does not limit the execution sequence of the two steps. The method 400 may begin at step 405.

Step 405: obtaining training data related to a health measurement of a user from a sensing device and corresponding timestamps. In one embodiment, the training data may be manually labeled or automatically labeled with initial preset timings. In an alternative embodiment, the training data may already include labeled samples. Each sample may include measurements and corresponding timestamps as well as an label of preferred timing of communication.

Step 410: based on the training data, performing or training a second machine learning algorithm/model to find the pattern between the timing of communication and the related measurements.

Step 415: sending out a question to a client computer for a user to confirm or reject the inferenced timings of communication with respect to the related measurements and corresponding timestamps. When the user confirms the inferenced timings, the flow proceeds to Step 420.

Step 420: applying a new confirmed second timing in the following communication with the client computer.

Step 425: collecting a second response from the client computer with respect to the second timing of communication. The second response may include a preferred timing of communication.

Step 430: refining the second machine learning algorithm/model with the second timing of the communication and the second response.

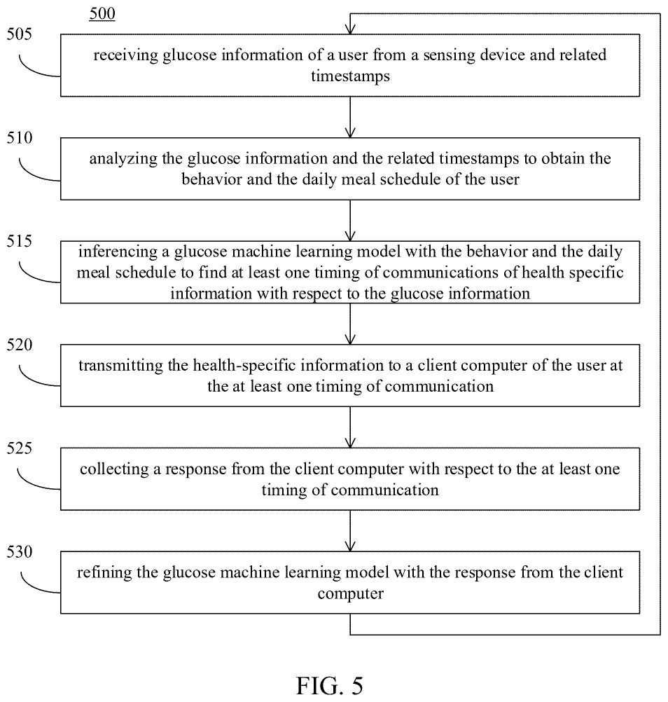

Please refer to FIG. 5, which illustrates a flowchart diagram of a method 500 for finding timings to transmit health specific information in accordance with an embodiment of the present application. The method 500 is a variant of the method 300 as shown in FIG. 3. This method 500 is configured to show an embodiment in case that the physiological information is glucose information of the user. The sensing device may be a CGM (Continuous Glucose Monitoring) device which tracks blood glucose levels all day long without finger pricks.

Similar to the method 300, the method 500 may be realized by the network system 100 as shown in FIG. 1. Especially, the method 500 may be embodied as computer instructions and data being executed by the server 110 as shown in FIG. 1. If no casual relation between two steps as shown in FIG. 5, the present application does not limit the execution sequence of the two steps. The method 500 may begin at step 505.

Step 505: receiving glucose information of a user from a sensing device and related timestamps. The glucose information may include one or more measurement of blood glucose level. After meal, the blood glucose level would raise. When the user is hungry, the blood glucose level would be low. Based on the blood glucose levels and associated timestamps, the server can tell when the user ate.

Step 510: analyzing the glucose information and the related timestamps to obtain the behavior and the daily meal schedule of the user. In one embodiment, the analysis may be based on an algorithm other than machine learning model. The algorithm can tell the daily meal schedule. In an alternative embodiment, the analysis may include one machine learning model which correlate the blood glucose levels and the associated timestamps with the daily meal schedule.

Step 515: inferencing a glucose machine learning model with the behavior and the daily meal schedule to find at least one timing of communication of health specific information with respect to the glucose information.

In one embodiment, the glucose machine learning model may provide a timing which is ahead of a meal. The timing may be corresponding to a diet related information such as diet preparation tips including less salt and avoiding sugary intake.

In an alternative embodiment, the glucose machine learning model may provide another timing which is after the meal. Another timing may be corresponding to an exercise suggestion such as taking a walk to help the user to meet his daily exercise plan.

In another embodiment, the glucose machine learning model may provide a timing of an after-meal report which is generated according to the physiological measurements of blood glucose levels after the meal. The user can see the glucose excursion in the after-meal report. Moreover, the dietitian of the user can use this after-meal report to adjust or fine tune the meal plan.

Step 520: transmitting the health-specific information to a client computer of the user at the at least one timing of communication. In one embodiment, the server may transmit the diet related information at the timing before the meal and also transmit the exercise suggestion at another timing after the meal.

Step 525: collecting a response from the client computer with respect to the at least one timing of communication.

Step 530: refining the glucose machine learning model with the response from the client computer.

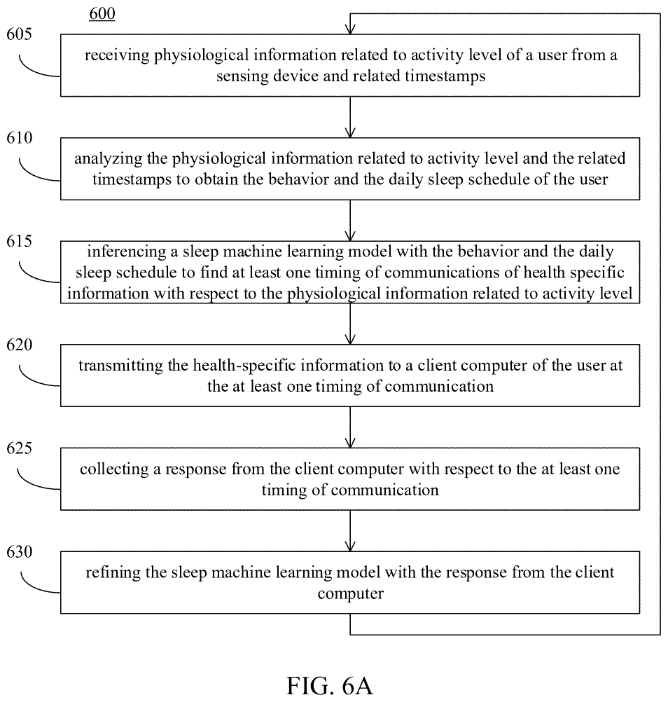

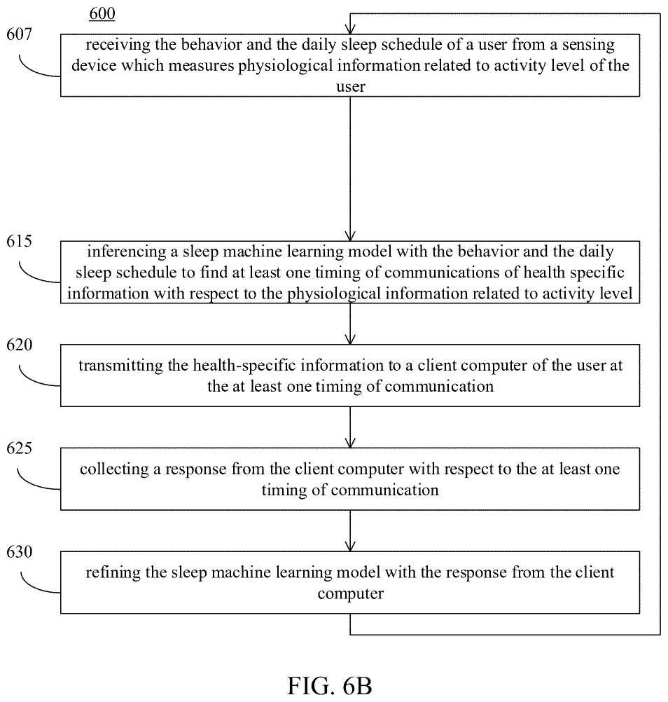

Please refer to FIG. 6A, which illustrates a flowchart diagram of a method 600 for finding timings to transmit health specific information in accordance with an embodiment of the present application. The method 600 is a variant of the method 300 as shown in FIG. 3. This method 600 is configured to show an embodiment in case that the physiological information is related to sleep information of the user.

In one embodiment, the sensing device may be a smart wearable device such as a smart ring, a smart watch, or a smart band. This kind of smart wearable devices may equip with an oximeter, a sphygmomanometer, a heartbeat rate sensor and/or a motion sensor. Therefore, the sensed physiological information can tell whether the user is fallen asleep or not. The determination of the sleep status of the user may be performed in the sensing device 130 or in the server 110. It means that the sensing device 130 may send a status indicating whether the user is sleeping or not to the server 110. Or the sensing device 130 may send the measured physiological information and motion information to the server 110 which determines whether the user is asleep or not based on the information sent by the sensing device 130.

Similar to the method 300, the method 600 may be realized by the network system 100 as shown in FIG. 1. Especially, the method 600 may be embodied as computer instructions and data being executed by the server 110 as shown in FIG. 1. If no casual relation between two steps as shown in FIG. 6A, the present application does not limit the execution sequence of the two steps. The method 600 may begin at step 605.

Step 605: receiving physiological information related to activity level of a user from a sensing device and related timestamps. In one embodiment, the physiological information may comprise the motion information of a body part, e.g., hand, wrist, or finger, of the user.

Step 610: analyzing the physiological information related to activity level and the related timestamps to obtain the behavior and the daily sleep schedule of the user.

Step 615: inferencing a sleep machine learning model with the behavior and the daily sleep schedule to find at least one timing of communications of health specific information with respect to the physiological information related to activity level.

In one embodiment, the sleep machine learning model may provide a timing of bedtime reminder before sleep. The bedtime reminder may include sleep related information such as sleep rituals and teeth brush. In an alternative embodiment, the sleep learning model may provide a timing of sleep quality questionnaire after wake up to collect the user's opinion of sleep quality. In addition to the sleep schedule, the sleep machine learning model may provide a timing of rest reminder or a timing of nap reminder to the user.

Step 620: transmitting the health-specific information to a client computer of the user at the at least one timing of communication. The health-specific information may be one of abovementioned bedtime reminder, rest reminder, nap reminder, and the wake-up questionnaire.

Step 625: collecting a response from the client computer with respect to the at least one timing of communication.

Step 630: refining the sleep machine learning model with the response from the client computer.

Please refer to FIG. 6B, which illustrates a variant of the flowchart diagram of the method 600 as shown in FIG. 6A. As shown in FIG. 6B, Steps 605 and 610 are replaced by Step 607. Other steps as shown in FIG. 6A are employed in the flowchart as shown in FIG. 6B. The method 600 as shown in FIG. 6B may begins at Step 607.

At Step 607, the server 110 is configured for receiving the behavior and the daily sleep schedule of a user from a sensing device which measures physiological information related to activity level of the user. In other words, the analysis is done by the sensing device 130, rather than the server 110.

In an example, a daily sleep schedule was outputted as an analyzed result. The daily sleep schedule of the user is from 10:00 pm to 7:00 am. Therefore, a timing of bedtime reminder provided by the sleep machine learning model is at 8:00 pm, i.e., two hours ahead of the bedtime. And another timing of wake-up questionnaire survey initially provided by the sleep machine learning model is at 7:15 am, i.e., 15 minutes after wake-up.

However, based on the user's responses, it seems the timing of wake-up questionnaire survey at 7:15 am is inconvenient for him. Thus, the sleep machine learning model adjust the timing from 7:15 am to 1:00 pm based on the user's responses.

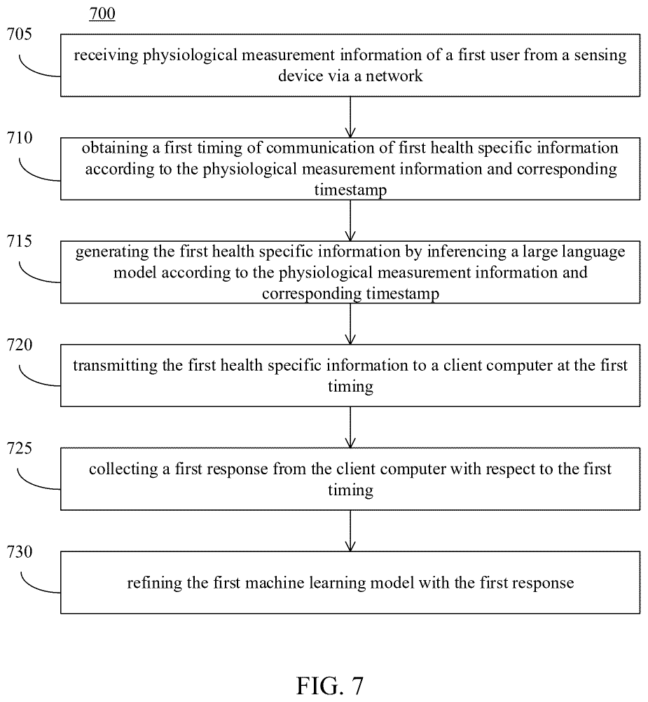

Please refer to FIG. 7, which illustrates a flowchart diagram of a method 700 for finding timings to transmit health specific information in accordance with an embodiment of the present application. The method 700 may be realized by the network system 100 as shown in FIG. 1. Especially, the method 700 may be embodied as computer instructions and data being executed by the server 110 as shown in FIG. 1. If no casual relation between two steps as shown in FIG. 7, the present application does not limit the execution sequence of the two steps. The method 700 may begin at step 705.

Step 705: receiving physiological measurement information of a first user from a sensing device via a network.

Step 710: obtaining a first timing of communication of first health specific information according to the physiological measurement information and corresponding timestamp. The timestamp may be received with the physiological measurement information or be generated locally. The first timing of communication may be an inference result of a first machine learning model.

Step 715: generating the first health specific information by inferencing a large language model according to the physiological measurement information and corresponding timestamp. Alternatively, the first health specific information may be generated by a deterministic algorithm according to the physiological measurement information and corresponding timestamp.

Step 720: transmitting the first health specific information to a client computer at the first timing. The client computer may be used by the user of the sensing device. Or the client computer may be used by another user who is a caretaker of the user of the sensing device. In one example, the sensing device and the client computer may be in different place. The sensing device may be in a room where the user stays. And the client computer may be in an office where the another user stays. The sensing device and the client computer connects to different access networks.

Step 725: collecting a first response from the client computer with respect to the first timing.

Step 730: refining the first machine learning model with the first response.

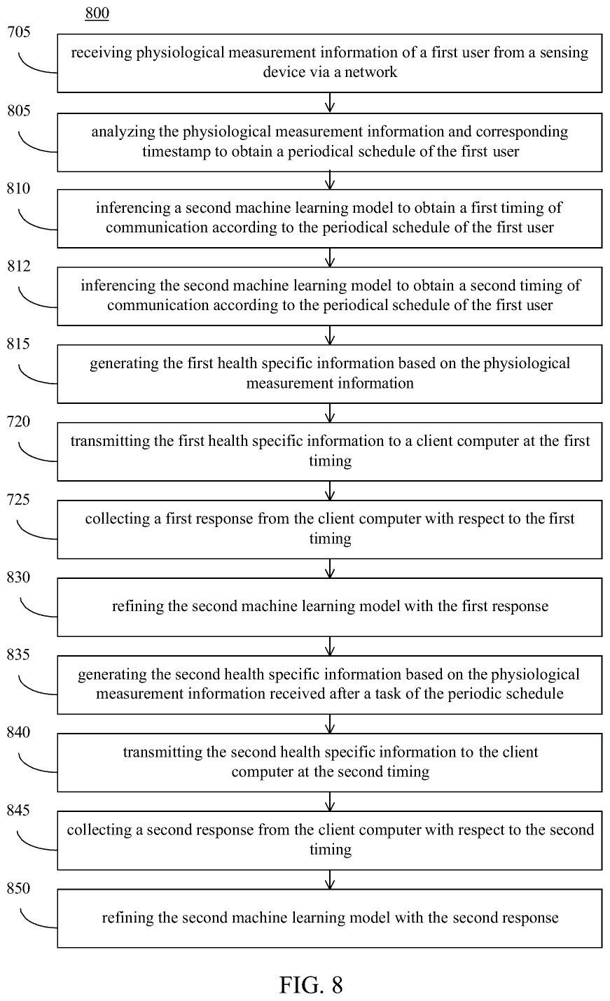

Please refer to FIG. 8, which depicts a method 800 for finding timings to transmit health specific information in accordance with an embodiment of the present application. The method 800 is a variant of the method 700. The method 800 may be realized by the network system 100 as shown in FIG. 1. Especially, the method 800 may be embodied as computer instructions and data being executed by the server 110 as shown in FIG. 1. If no casual relation between two steps as shown in FIG. 8, the present application does not limit the execution sequence of the two steps.

Some steps of the method 700 are reused in the method 800. The method 800 may begin at Step 705. After Step 705, the flow may proceed to Step 805.

Step 805: analyzing the physiological measurement information and corresponding timestamp to obtain a periodical schedule of the first user. The periodical schedule may be a daily sleep schedule, a daily meal schedule, a weekly exercise schedule. Each of the periodical schedule comprises at least one task and its time period, such as a sleep time, breakfast, lunch, dinner, a morning jog etc. Next, the flow may proceed to Step 810 or Step 812.

Step 810: inferencing a second machine learning model to obtain a first timing of communication according to the periodical schedule of the first user. The second machine learning model as shown in FIG. 8 may be different from the first machine learning model as shown in FIG. 7 because its input is the analysis result, i.e., the periodic schedule of the user, rather than the physiological measurements and corresponding timestamps.

In one embodiment, the first timing of communication may be corresponding to a reminder of a task of the periodic schedule. For example, a sleep ritual ahead of a bedtime; or a diet plan to be presented before a meal. Next, the flow may proceed to Step 812 or Step 815.

Step 812: inferencing the second machine learning model to obtain a second timing of communication according to the periodical schedule of the first user. In one embodiment, the second timing of communication may be corresponding to an action, or a report of physiological measurement information received after at least one task of the periodic schedule. For example, a glucose excursion after a meal; or a sleep quality survey after wake up. Next the flow may proceed to Step 815.

Optionally, the flow may proceed to Step 720, Step 725, and Step 830. Step 830: refining the second machine learning model with the first response. In case the second timing of communication is obtained at Step 812, the flow may further proceed to Step 835, Step 840, Step 845, and Step 850.

Step 835: generating the second health specific information based on the physiological measurement information received during or after a task of the periodic schedule. For example, the glucose excursion report including the blood glucose levels measured during or after a meal must be generated after the task/meal.

Step 840: transmitting the second health specific information to the client computer at the second timing.

Step 845: collecting a second response from the client computer with respect to the second timing.

Step 850: refining the second machine learning model with the second response.

According to an embodiment of the present application, a method for finding a timing of communication with respect to physiological information and associated timestamps is provided. The method comprising: receiving physiological measurement information of a first user from a sensing device via a network; obtaining a first timing of communication of first health specific information according to the physiological measurement information and corresponding timestamp; and transmitting the first health specific information to a client computer at the first timing. The corresponding timestamp may be one of following: a timestamp of uploading the physiological measurement information in the sensing device or a timestamp of receiving the physiological measurement information in the server.

Preferably, in order to prepare the first health specific information, the method further comprises: generating the first health specific information by inferencing a large language model according to the physiological measurement information and corresponding timestamp.

Preferably, in order to realize a remote sensing, wherein the client computer is operated by a second user, the sensing device and the client computer connects to different access networks which are parts of the network, respectively.

Preferably, in order to take advantage of artificial intelligence, said obtaining step further comprises: inferencing a first machine learning model to obtain the first timing of communication according to the physiological measurement information and the corresponding timestamp.

Preferably, in order to fit the user's need, the method further comprises: collecting a first response from the client computer with respect to the first timing via the network; and refining the first machine learning model with the first response.

Preferably, in order to correlate timing with the task, said obtaining step further comprises: analyzing the physiological measurement information and the corresponding timestamp to obtain a periodical schedule of the first user; and inferencing a second machine learning model to obtain the first timing of communication according to the periodical schedule of the first user.

Preferably, in order to fit the user's need, the method further comprises: collecting a first response from the client computer with respect to the first timing via the network; and refining the second machine learning model with the first response.

Preferably, in order to take advantage of artificial intelligence, said analyzing step further comprises: inferencing a third machine learning model to obtain the periodical schedule of the first user according to the physiological measurement information and the corresponding timestamp.

Preferably, in order to provide reminder for incoming task, wherein the periodical schedule comprises at least one task, the first timing of communication is ahead the at least one task, wherein the first health-specific information is configured to remind the first user an action before the at least one task.

Preferably, in order to provide a report of the passed task, the method further comprises: obtaining a second timing of communication of second health specific information according to the periodic schedule; generating the second health specific information based on the physiological measurement information which is received during or after the at least one task; and transmitting the health specific information to the client computer at the second timing, the second timing of communication is after the at least one task.

In one embodiment of the present application, a server computer for finding a timing of communication with respect to physiological information and associated timestamps is provided. The server computer comprising: a networking device connected to a sensing device and a client computer via a network; and a processor configured for executing instructions stored in non-volatile memory for realizing following: having the networking device receive physiological measurement information of a first user from the sensing device; obtaining a first timing of communication of first health specific information according to the physiological measurement information and the corresponding timestamp; and having the networking device transmit the health specific information to a client computer at the first timing.

Preferably, in order to prepare the first health specific information, the processor is further configured for generating the first health specific information by inferencing a large language model according to the physiological measurement information and corresponding timestamp.

Preferably, in order to realize a remote sensing, wherein the client computer is operated by a second user, the sensing device and the client computer connects to different access networks which are parts of the network, respectively.

Preferably, in order to take advantage of artificial intelligence, said obtaining further comprises inferencing a first machine learning model to obtain the first timing of communication according to the physiological measurement information and the corresponding timestamp.

Preferably, in order to fit the user's need, wherein the processor is further configured for collecting a first response from the client computer with respect to the first timing via the network; and refining the first machine learning model with the first response.

Preferably, in order to correlate timing with the task, said obtaining further comprises: analyzing the physiological measurement information and the corresponding timestamp to obtain a periodical schedule of the first user; and inferencing a second machine learning model to obtain the first timing of communication according to the periodical schedule of the first user.

Preferably, in order to fit the user's need, the processor is further configured for collecting a first response from the client computer with respect to the first timing via the network; and refining the second machine learning model with the first response.

Preferably, in order to take advantage of artificial intelligence, said analyzing further comprises: inferencing a third machine learning model to obtain the periodical schedule of the first user according to the physiological measurement information and the corresponding timestamp.

Preferably, in order to provide reminder for incoming task, wherein the periodical schedule comprises at least one task, the first timing of communication is ahead the at least one task, wherein the first health-specific information is configured to remind the first user an action before the at least one task.

Preferably, in order to provide a report of the passed task, the processor is further configured for: obtaining a second timing of communication of second health specific information according to the periodic schedule; generating the second health specific information based on the physiological measurement information which is received during or after the at least one task; and transmitting the health specific information to the client computer at the second timing, the second timing of communication is after the at least one task.

According to an embodiment of the present application, a network system for finding a timing of communication with respect to physiological information and associated timestamps is provided. The network system comprises the server, the sensing device, and the client computer.

Based on the method and the server provided in the present application, an instruction informing mechanism for notifying patients or users at appropriate timings to meet their feasible options and to respect their decisions are presented. The mechanism can automatically analyze the periodic schedule and timings of communication based on the physiological measurement information and corresponding timestamps. Hence, health specific information such as reminder, an action suggestion, and/or a summary of physiological measurement information can be informed at the timings of communication in which the mechanism finds. It helps the patients or the users to follow the instructions in the timings of communication which do not conflict with other activities of the user. And the responsive report of physiological measurement information can be delivered to the patients or the users in appropriate timings. Following this mechanism, the user may make a better choice proactively and reflectively because the mechanism provide right information to the user at the right time.

While the invention has been described in terms of what is presently considered to be the most practical and preferred embodiments, it is to be understood that the invention needs not to be limited to the above embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for finding a timing of communication with respect to physiological information and associated timestamps, comprising:
receiving physiological measurement information of a first user from a sensing device via a network;
obtaining a first timing of communication of first health specific information according to the physiological measurement information and corresponding timestamp; and
transmitting the first health specific information to a client computer at the first timing.

2. The method as recited in claim 1, further comprises: generating the first health specific information by inferencing a large language model according to the physiological measurement information and corresponding timestamp.

3. The method as recited in claim 1, wherein the client computer is operated by a second user, the sensing device and the client computer connects to different access networks which are parts of the network, respectively.

4. The method as recited in claim 1, wherein said obtaining step further comprises: inferencing a first machine learning model to obtain the first timing of communication according to the physiological measurement information and the corresponding timestamp.

5. The method as recited in claim 4, further comprises:
collecting a first response from the client computer with respect to the first timing via the network; and
refining the first machine learning model with the first response.

6. The method as recited in claim 1, wherein said obtaining step further comprises:
analyzing the physiological measurement information and the corresponding timestamp to obtain a periodical schedule of the first user; and
inferencing a second machine learning model to obtain the first timing of communication according to the periodical schedule of the first user.

7. The method as recited in claim 6, further comprises:
collecting a first response from the client computer with respect to the first timing via the network; and
refining the second machine learning model with the first response.

8. The method as recited in claim 6, further comprises:
inferencing a third machine learning model to obtain the periodical schedule of the first user according to the physiological measurement information and the corresponding timestamp.

9. The method as recited in claim 6, wherein the periodical schedule comprises at least one task, the first timing of communication is ahead the at least one task, wherein the first health-specific information is configured to remind the first user an action before the at least one task.

10. The method as recited in claim 9, further comprises:
obtaining a second timing of communication of second health specific information according to the periodic schedule;
generating the second health specific information based on the physiological measurement information which is received during or after the at least one task; and
transmitting the health specific information to the client computer at the second timing, the second timing of communication is after the at least one task.

11. A server computer for finding a timing of communication with respect to physiological information and associated timestamps, wherein the server computer comprising:
a networking device connected to a sensing device and a client computer via a network; and
a processor configured for executing instructions stored in non-volatile memory for realizing following:
having the networking device receive physiological measurement information of a first user from the sensing device;
obtaining a first timing of communication of first health specific information according to the physiological measurement information and the corresponding timestamp; and
having the networking device transmit the health specific information to a client computer at the first timing.

12. The server computer as recited in claim 11, wherein the processor is further configured for generating the first health specific information by inferencing a large language model according to the physiological measurement information and corresponding timestamp.

13. The server computer as recited in claim 11, wherein the client computer is operated by a second user, the sensing device and the client computer connects to different access networks which are parts of the network, respectively.

14. The server computer as recited in claim 11, wherein said obtaining further comprises inferencing a first machine learning model to obtain the first timing of communication according to the physiological measurement information and the corresponding timestamp.

15. The server computer as recited in claim 14, wherein the processor is further configured for:
collecting a first response from the client computer with respect to the first timing via the network; and
refining the first machine learning model with the first response.

16. The server computer as recited in claim 11, wherein said obtaining further comprises:
analyzing the physiological measurement information and the corresponding timestamp to obtain a periodical schedule of the first user; and
inferencing a second machine learning model to obtain the first timing of communication according to the periodical schedule of the first user.

17. The server computer as recited in claim 16, wherein the processor is further configured for:
collecting a first response from the client computer with respect to the first timing via the network; and
refining the second machine learning model with the first response.

18. The server computer as recited in claim 16, wherein said analyzing further comprises:
inferencing a third machine learning model to obtain the periodical schedule of the first user according to the physiological measurement information and the corresponding timestamp.

19. The server computer as recited in claim 16, wherein the periodical schedule comprises at least one task, the first timing of communication is ahead the at least one task, wherein the first health-specific information is configured to remind the first user an action before the at least one task.

20. The server computer as recited in claim 19, wherein the processor is further configured for:
obtaining a second timing of communication of second health specific information according to the periodic schedule;
generating the second health specific information based on the physiological measurement information which is received during or after the at least one task; and
transmitting the health specific information to the client computer at the second timing,
wherein the second timing of communication is after the at least one task.

21. A network system for finding a timing of communication with respect to physiological information and associated timestamps, wherein the network system comprising the server, the sensing device, and the client computer as recited in claim 11.
